(19) United States
(12) Patent Application Publication
(10) Pub. No.: US 2010/0139133 A1
(43) Pub. Date: Jun. 10, 2010

(54) ELASTIC TAGS

(76) Inventor: Tammy C. Adams, Columbus, MS (US)

Correspondence Address: WITHERS & KEYS, LLC, P. O. BOX 2049, MCDONOUGH, GA, 30253, US

(21) Appl. No.: 12/331,913

(22) Filed: Dec. 10, 2008

(51) Int. Cl. G09F 3/00; A61B 19/10

(52) U.S. Cl. 40/299.01; 128/849

(57) ABSTRACT

Elastic tags, method of making elastic tags, and methods of using elastic tags are disclosed.

### TECHNICAL FIELD

The present invention relates to elastic tags and methods of making and using the same.

### BACKGROUND OF THE INVENTION

There is a need in the art for elastic tags that are (i) suitable for a variety of uses including, but not limited to, binding one or more articles to one or more other articles or to itself, and identifying merchandise, (ii) easy to use, and (iii) relatively inexpensive to manufacture.

### SUMMARY OF THE INVENTION

The present invention is directed to elastic tags, method of making elastic tags, and methods of using elastic tags. The elastic tags of the present invention may be used in a variety of applications, and are particularly useful in the medical industry for controlling the position of a drape relative to, for example, a piece of equipment (e.g., a microscope).

In one exemplary embodiment, the elastic tag of the present invention comprises a first tag portion having an outer periphery extending along an outer edge of the first tag portion; and a second tag portion integrally attached to the first tag portion and extending beyond a first edge portion of the outer edge of the first tag portion, the second tag portion comprising a first closed loop having a loop inner periphery, at least a portion of the loop inner periphery being positioned a distance from the first tag portion and comprising an elastomeric material, wherein the first tag portion further comprises (a) at least one slotted opening positioned along and in communication with the outer periphery, (b) a second closed loop of elastic material, or (c) both (a) and (b).

The present invention is further directed to methods of making and using the disclosed elastic tags. In one exemplary method of making an elastic tag, the method comprises integrally attaching a second tag portion to a first tag portion so that the second tag portion extends beyond a first edge portion of an outer edge of the first tag portion, the second tag portion comprising a first closed loop having a loop inner periphery, at least a portion of the loop inner periphery being positioned a distance from the first tag portion and comprising an elastomeric material; and at least one step selected from the steps of: (1) forming at least one slotted opening within the first tag portion so as to be in communication with an outer periphery of the first tag portion, and (2) providing a second closed loop of elastic material along the outer periphery of the first tag portion.

In one exemplary method of using an elastic tag, the method comprises surrounding at least a portion of an article with an elastic tag comprising a first tag portion having a first closed loop integrally attached thereto; and connecting the first closed loop of the elastic tag with (i) one or more slotted opening within the first tag portion or (ii) a second closed loop of elastic material along an outer periphery of the first tag portion. In one exemplary embodiment, the article comprises a drape, and the method is used to secure at least a portion of the drape to a piece of medical equipment such as a microscope.

These and other features and advantages of the present invention will become apparent after a review of the following detailed description of the disclosed embodiments and the appended claims.

### BRIEF DESCRIPTION OF THE DRAWINGS

The present invention is further described with reference to the appended figures, wherein:

FIGS. 1A-1D depict frontal views of exemplary elastic tags of the present invention;

FIGS. 2A-2D depict exemplary cross-sectional views of the exemplary elastic tag shown in FIG. 1A as viewed along line A-A as shown in FIG. 1A;

FIGS. 3A-3C depict frontal and side views of the exemplary elastic tags of FIGS. 1A and 1C when a closed loop portion of a given exemplary elastic tag is connected to at least one slotted opening of the given exemplary elastic tag;

FIGS. 4A-4B depict frontal views of other exemplary elastic tags of the present invention; and

FIG. 5 depicts a side view of the exemplary elastic tag of FIG. 4A when a first closed loop portion of the exemplary elastic tag is connected to a second closed loop portion of the exemplary elastic tag via a connection member.

### DETAILED DESCRIPTION OF EXEMPLARY EMBODIMENTS

Although the present invention is herein described in terms of specific embodiments, it will be readily apparent to those skilled in this art that various modifications, rearrangements, and substitutions can be made without departing from the spirit of the invention. The scope of the present invention is thus only limited by the claims appended hereto.

The present invention is directed to elastic tags. The elastic tags of the present invention comprise a first tag portion having an outer periphery extending along an outer edge of the first tag portion, and a second tag portion integrally attached to the first tag portion and extending beyond a first edge portion of the outer edge of the first tag portion. The second tag portion comprises a first closed loop having a loop inner periphery. At least a portion of the loop inner periphery is positioned a distance from the first tag portion and comprising an elastomeric material. The first tag portion further comprises (a) at least one slotted opening positioned along and in communication with the outer periphery, (b) a second closed loop of elastic material, or (c) both (a) and (b).

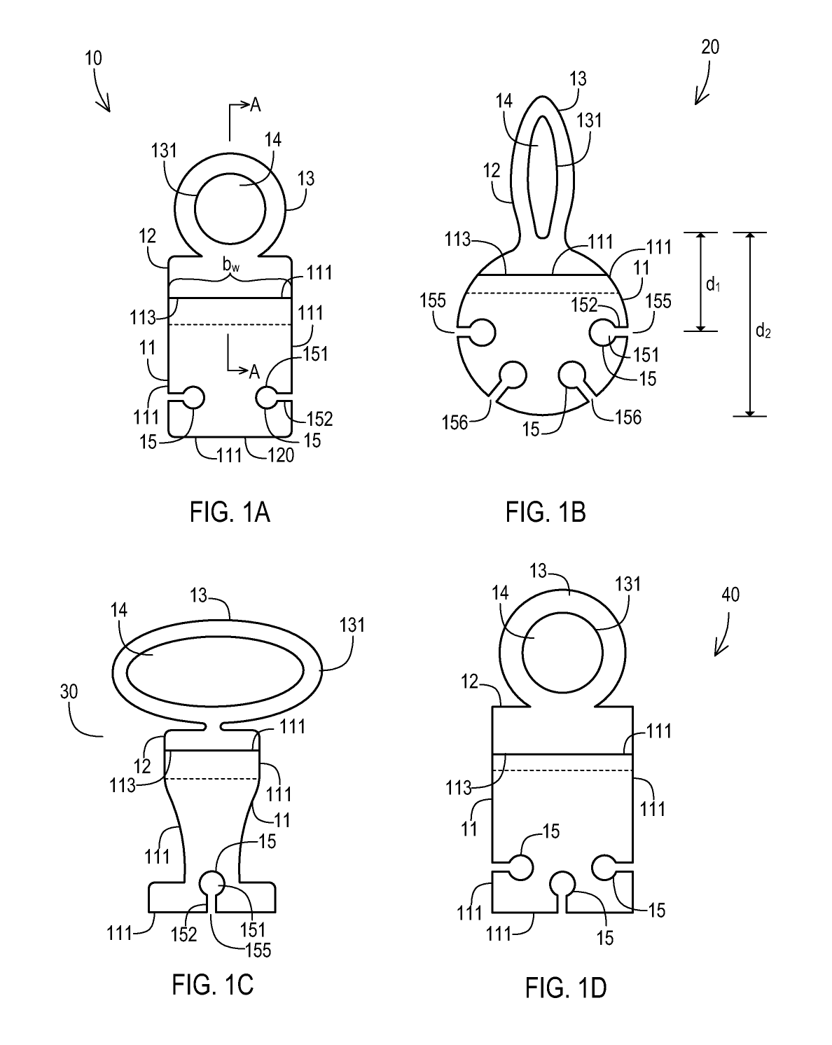

Exemplary elastic tags of the present invention are shown in FIGS. 1A-1D. As shown in FIG. 1A, exemplary elastic tag 10 comprises a first tag portion 11 having an outer periphery 111 extending along most of an outer edge 112 of first tag portion 11. Exemplary elastic tag 10 further comprises a second tag portion 12 integrally attached to first tag portion 11. As shown in FIG. 1A, second tag portion 12 extends beyond a first edge portion 113 of the outer edge 112 of first tag portion 11, and comprises a first closed loop 13 having a loop inner periphery 131 that surrounds a first closed loop opening 14.

Exemplary elastic tag 10 further comprises at least one slotted opening 15 positioned along and in communication with outer periphery 111. As used herein, the phrase “in communication with” refers to outer periphery 111 and a given slotted opening 15 sharing a common edge, namely, outer edge 112 of first tag portion 11 (i.e., the portion of outer edge 112 of first tag portion 11 that extends into and forms an inner edge of a given slotted opening 15). Each slotted opening 15 comprises a loop containment reservoir 151 and a channel 152 that connects loop containment reservoir 151 with outer periphery 111.

As used herein, outer periphery 111 is used to describe an outer perimeter extending along first tag portion 11 and includes (i) portions of outer edge 112 and (ii) any gaps within outer edge 112 due to the presence of one or more channels 152 along outer periphery 111. In contrast, as discussed above, outer edge 112 is used to describe a continuous outer edge of first tag portion 11 that includes (i) portions of outer edge 112 extending along outer periphery 111 and (ii) inner edge portions of any slotted openings 15 (i.e., inner edge portions of one or more channels 152 and inner edge portions of one or more loop containment reservoirs 151 positioned within outer periphery 111.

Another exemplary elastic tag 20 is shown in FIG. 1B. Exemplary elastic tag 20 comprises a first tag portion 11 having an outer periphery 111 extending along an outer edge (not shown) of first tag portion 11. Exemplary elastic tag 20 further comprises a second tag portion 12 integrally attached to first tag portion 11. As shown in FIG. 1B, second tag portion 12 extends beyond a first edge portion 113 of the outer edge 112 of first tag portion 11, and comprises a first closed loop 13 having a loop inner periphery 131 that surrounds first closed loop opening 14. In addition, exemplary elastic tag 20 comprises a pair of slotted openings 15 positioned along and in communication with outer periphery 111. As shown in FIG. 1B, a first pair 155 of slotted openings 15 is positioned a substantially equal first distance d₁ from first closed loop 13, and a second pair 156 of slotted openings 15 is positioned a substantially equal second distance d₂ from first closed loop 13.

A further exemplary elastic tag 30 is shown in FIG. 1C. Exemplary elastic tag 30 comprises a first tag portion 11 having an outer periphery 111 extending along an outer edge (not shown) of first tag portion 11. Exemplary elastic tag 30 further comprises a second tag portion 12 integrally attached to first tag portion 11. As shown in FIG. 1C, second tag portion 12 extends beyond a first edge portion 113 of the outer edge 112 of first tag portion 11, and comprises a first closed loop 13 having a loop inner periphery 131 that surrounds first closed loop opening 14. In this embodiment, exemplary elastic tag 30 comprises a single slotted opening 15 positioned along and in communication with outer periphery 111.

Yet another exemplary elastic tag 40 is shown in FIG. 1D. Exemplary elastic tag 40 comprises a first tag portion 11 having an outer periphery 111 extending along an outer edge (not shown) of first tag portion 11. Exemplary elastic tag 40 further comprises a second tag portion 12 integrally attached to first tag portion 11. As shown in FIG. 1D, second tag portion 12 extends beyond a first edge portion 113 of the outer edge 112 of first tag portion 11, and comprises a first closed loop 13 having a loop inner periphery 131 that surrounds first closed loop opening 14. In this embodiment, exemplary elastic tag 40 comprises three slotted openings 15 positioned along and in communication with outer periphery 111.

As shown in FIGS. 1A-1D, elastic tags of the present invention may have one or more slotted openings 15 positioned along and in communication with outer periphery 111. In one exemplary embodiment, outer periphery 111 of first tag portion 11 comprises (i) a second edge portion (e.g., second edge portion 120 shown in FIG. 1A) opposite a first edge portion (e.g., first edge portion 113), (ii) opposite side edges connecting the second edge portion to the first edge portion, and (iii) at least one slotted opening 15 positioned along each of the opposite side edges. In another exemplary embodiment, outer periphery 111 of first tag portion 11 comprises (i) a second edge portion (e.g., second edge portion 120 shown in FIG. 1A) opposite a first edge portion (e.g., first edge portion 113), (ii) opposite side edges connecting the second edge portion to the first edge portion, (iii) at least one slotted opening positioned along the second edge portion, and (iv) at least one slotted opening positioned along one or both of the opposite side edges.

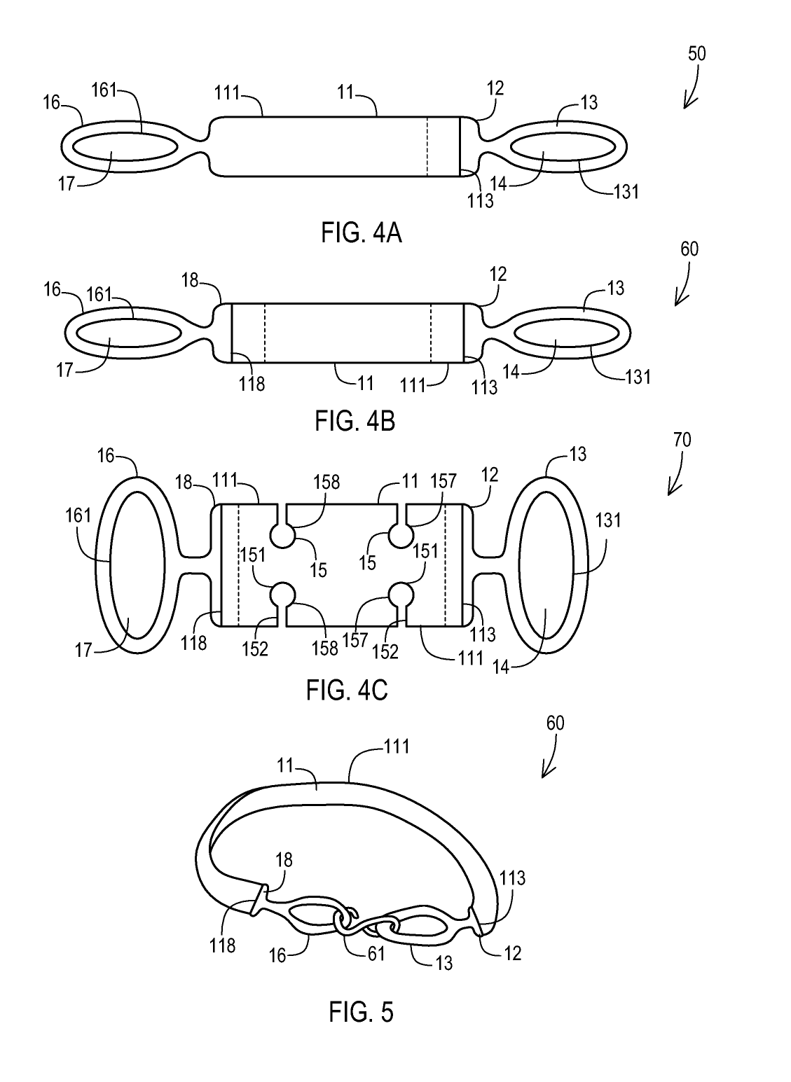

In each of the exemplary elastic tags shown in FIGS. 1A-1D (and in FIG. 4B described below), it should be understood that each slotted opening 15 may independently have any desired shape, size and configuration as long as the slotted opening 15 is positioned within first tag portion 11 and in communication with outer periphery 111. For example, although each loop containment reservoir 151 is shown as having a circular shape, each loop containment reservoir 151 may independently have any desired shape including, but not limited to, a triangular shape, a square shape, a rectangular shape, a diamond shape, a polygonal shape, a hexagonal shape, a trapezoidal shape, an oval shape, an oblong shape, an irregular shape (e.g., a “FIG. 8” shape), or any other desired shape. Further, each loop containment reservoir 151 may independently have any desired size. Typically, each loop containment reservoir 151 has a largest dimension (e.g., diameter, length, width, etc.) that is less than an overall width W of first tag portion 11, more typically, less than half of an overall width of first tag portion 11 (i.e., ½W). In one exemplary embodiment, each loop containment reservoir 151 has a largest dimension (e.g., diameter, length, width, etc.) that is less than about 25.4 millimeters (mm) (1.0 inches (in.)), typically, less than about 12.7 mm (0.5 in.), and more typically, between about 0.64 mm (0.25 in.) to about 12.7 mm (0.5 in.).

In addition, each channel 152 may independently have any desired shape, size and configuration. For example, although each channel 152 is shown as having a rectangular shape with a channel width and a channel length greater than the channel width, each channel 152 may independently have any desired shape including, but not limited to, a square shape, a diamond shape, a polygonal shape, a trapezoidal shape, an irregular shape (e.g., an “S” shape), or any other desired shape. Further, each channel 152 may independently have any desired dimensions. Typically, each channel 152 has a channel length greater than a channel width. Further, typically, the channel width remains substantially constant along the channel length. In one exemplary embodiment, each channel 152 has a channel width that is less than about 12.7 mm (0.5 in.), typically, less than about 0.64 mm (0.25 in.), and more typically, between about 0.01 mm (0.4 mils) to about 0.64 mm (0.25 in.); and a channel length that is less than about 25.4 mm (1.0 in.), typically, less than about 12.7 mm (0.5 in.), and more typically, between about 0.64 mm (0.25 in.) to about 12.7 mm (0.5 in.).

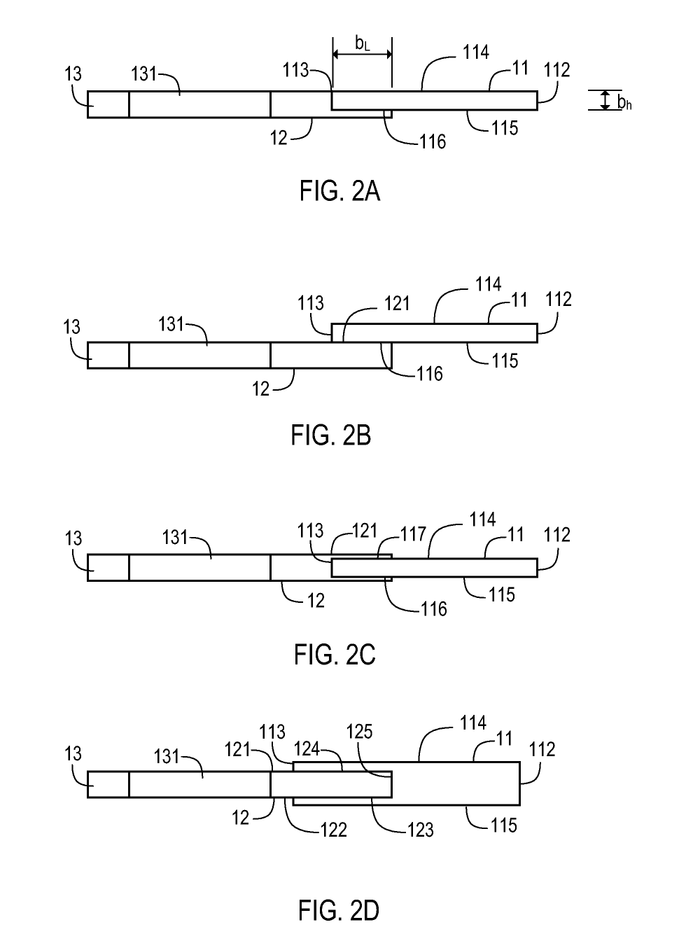

In each of the exemplary elastic tags shown in FIGS. 1A-1D (and in FIG. 4B described below), first tag portion 11 may be integrally attached to second tag portion 12 using a variety of bond configurations such as the exemplary bond configurations shown in FIGS. 2A-2D. FIGS. 2A-2D depict exemplary cross-sectional views of the exemplary elastic tag shown in FIG. 1A as viewed along line A-A as shown in FIG. 1A. As shown in FIG. 2A, first tag portion 11 is integrally attached to second tag portion 12 via an exemplary bond configuration wherein a portion of first tag portion 11 is embedded within second tag portion 12. In this exemplary embodiment, first tag portion 11 has an upper surface 114 and a lower surface 115. A portion 116 of lower surface 115 and outer edge 112, in particular, first edge portion 113, is in contact with and integrally attached to second tag portion 12 along a bond length b_{L} and a bond height bₕ as shown in FIG. 2A, and a bond width b_{w} (see, for example, bond width b_{w} shown in FIG. 1A). Bond width b_{w}, bond length b_{L} and bond height bₕ may have dimensions that vary depending on a number of factors including, but not limited to, the desired degree of bond strength between first tag portion 11 and second tag portion 12, the materials used to form first tag portion 11 and second tag portion 12, and the overall dimensions of the elastic tag.

FIG. 2B provides another exemplary bond configuration. In this exemplary embodiment, first tag portion 11 is integrally attached to second tag portion 12 via an exemplary bond configuration wherein portion 116 of lower surface 115 is in contact with and integrally attached to an upper surface 121 of second tag portion 12 along a bond length b_{L} and a bond width b_{w}. As discussed above, bond width b_{w} and bond length b_{L} may have dimensions that vary depending on a number of factors.

FIG. 2C provides yet another exemplary bond configuration. As shown in FIG. 2C, first tag portion 11 is integrally attached to second tag portion 12 via an exemplary bond configuration wherein a portion of first tag portion 11 is completely embedded within second tag portion 12. A portion 116 of lower surface 115, first edge portion 113, and a portion 117 of upper surface 114 of first tag portion 11 is in contact with and integrally attached to second tag portion 12 along two bond length b_{L} (i.e., extending along portions 116 and 117) and a bond height bₕ (i.e., extending along first edge portion 113) as shown in FIG. 2C, and two bond width b_{w} (i.e., extending along portions 116 and 117). As discussed above, bond widths b_{w}, bond lengths b_{L} and bond height bₕ may have dimensions that vary depending on a number of factors.

In another exemplary bond configuration shown in FIG. 2D, first tag portion 11 is integrally attached to second tag portion 12 via an exemplary bond configuration wherein a portion of second tag portion 12 is completely embedded within first tag portion 11. In this exemplary embodiment, second tag portion 12 has an upper surface 121 and a lower surface 122. A portion 123 of lower surface 122, an edge portion 125, and a portion 124 of upper surface 121 of second tag portion 12 is in contact with and integrally attached to first tag portion 11 along two bond length b_{L} (i.e., extending along portions 123 and 124) and a bond height bₕ (i.e., extending along edge portion 125) as shown in FIG. 2D, and two bond width b_{w} (i.e., extending along portions 123 and 124). As discussed above, bond widths b_{w}, bond lengths b_{L} and bond height bₕ may have dimensions that vary depending on a number of factors.

As discussed above, some elastic tags of the present invention may comprise a first tag portion that further comprises a second closed loop of elastic material. Exemplary elastic tags of the present invention comprising a second closed loop of elastic material are shown in FIGS. 4A-4C.

As shown in FIG. 4A, exemplary elastic tag 50 comprises a first tag portion 11 having an outer periphery 111 extending along an outer edge (not shown) of first tag portion 11. Exemplary elastic tag 50 further comprises a second tag portion 12 integrally attached to first tag portion 11. As shown in FIG. 4A, second tag portion 12 extends beyond first edge portion 113 of first tag portion 11, and comprises first closed loop 13 having loop inner periphery 131 that surrounds first closed loop opening 14. Exemplary elastic tag 50 further comprises a second closed loop 16 having a loop inner periphery 161 that surrounds a second closed loop opening 17. In this exemplary embodiment, second closed loop 16 is an integral component of first tag portion 11. In other words, second closed loop 16 and first tag portion 11 are formed from the same piece of material (e.g., an elastic film sheet).

Another exemplary elastic tag 60 is shown in FIG. 4B. Exemplary elastic tag 60 comprises first tag portion 11 and second tag portion 12 integrally attached to first tag portion 11. As shown in FIG. 4B, second tag portion 12 extends beyond first edge portion 113 of first tag portion 11, and comprises first closed loop 13 having loop inner periphery 131 that surrounds first closed loop opening 14. In this exemplary embodiment, exemplary elastic tag 60 further comprises a third tag portion 18 integrally attached to first tag portion 11. As shown in FIG. 4B, third tag portion 18 extends beyond another edge portion 118 of the outer edge of first tag portion 11, and comprises second closed loop 16 having a loop inner periphery 161 that surrounds a second closed loop opening 17. At least a portion of loop inner periphery 161 is positioned a distance from first tag portion 11, and as shown in FIG. 4B, all of loop inner periphery 161 may be positioned a distance from first tag portion 11. Desirably, at least a portion (and possibly all) of loop inner periphery 161 comprises an elastomeric material.

Yet another exemplary elastic tag 70 is shown in FIG. 4C. Exemplary elastic tag 70 comprises first tag portion 11, second tag portion 12 integrally attached to first tag portion 11, and third tag portion 18 integrally attached to first tag portion 11. In this exemplary embodiment, exemplary elastic tag 70 further comprises a pair of slotted openings 15 positioned along and in communication with outer periphery 111 of first tag portion 11. As shown in FIG. 4C, a first pair 157 of slotted openings 15 is positioned a substantially equal first distance d₁ from first closed loop 13, and a second pair 158 of slotted openings 15 is positioned a substantially equal second distance d₂ from first closed loop 13.

In the exemplary embodiments shown in FIGS. 4A-4C, first tag portion 11 may be integrally bonded to second tag portion 12 and third tag portion 18 using any of the above-described bond configurations. As discussed above, the degree of bonding between (i) first tag portion 11 and (ii) second tag portion 12 and/or third tag portion 18 may be varied as desired, for example, by adjusting bond width(s) b_{w}, bond length(s) b_{L} and bond height bₕ. In some embodiments, it may be desirable for the bond strength between first tag portion 11 and second tag portion 12 to be greater than the bond strength between first tag portion 11 and third tag portion 18. In other embodiments, it may be desirable for the bond strength between first tag portion 11 and second tag portion 12 to be less than the bond strength between first tag portion 11 and third tag portion 18. In yet other embodiments, it may be desirable for the bond strength between first tag portion 11 and second tag portion 12 to be substantially equal to the bond strength between first tag portion 11 and third tag portion 18.

The elastic tags of the present invention may be formed from a variety of materials. For example, first tag portion 11, second tag portion 12 and third tag portion 18 may each independently comprise an elastomeric material. Suitable elastomeric materials include, but are not limited to, styrene-containing block copolymers (e.g., styrene-butadiene-styrene copolymers, styrene-isoprene-styrene copolymers, and styrene-ethylene-butylene-styrene copolymers), ethylene-propylene copolymers, natural rubbers, etc.

In some exemplary embodiments, first tag portion 11 comprises a sheet of inelastic material while second tag portion 12 and third tag portion 18 (when present) comprise any of the above-mentioned elastomeric materials. Typically, a substantial portion (i.e., greater than 50 wt-%) of second tag portion 12 and third tag portion 18 (when present) comprises an elastomeric material. More typically, at least 90 wt-% (or at least 95 wt-%, or at least 98 wt-%) of second tag portion 12 and third tag portion 18 (when present) comprises an elastomeric material.

Suitable inelastic materials may include, but are not limited to, paper, a polymeric film material, a fiber-reinforced polymeric film material, a polymeric foam material, an electrically conductive material, a ceramic material, a glass material, and any combinations thereof. When multiple inelastic materials are used in combination, first tag portion 11 may comprise a single layer of inelastic materials or multiple layers of inelastic materials. For example, first tag portion 11 may comprise a single sheet of paper or polymeric film, a single sheet of fiber-reinforced polymeric film material, or a multi-layered structure comprising a paper layer and one or more outer polymeric film layers on one or more major outer surfaces of the paper layer.

In one desired embodiment, first tag portion 11 comprises a sheet of material (i.e., a single layer or multi-layer sheet of material) having opposite outer major surfaces (e.g., upper surface 114 and lower surface 115), wherein at least one of the opposite outer major surfaces (e.g., upper surface 114 and lower surface 115) is capable of accepting and displaying indicia thereon. For example, in some embodiments, it may be advantageous to be able to print indicia on an outer surface of first tag portion 11, for example, using a laser or inkjet printer. In some embodiments, it may be advantageous to be able to write indicia on an outer surface of first tag portion 11, for example, using a pen, pencil or marker.

Various additives may be included in the materials used to form first tag portion 11, second tag portion 12 and/or third tag portion 18 of the elastic tags of the present invention. These additives may be, for example, added to a polymeric blend melt or added to the formed material after casting/molding (e.g., during a coating step). Such additives include, but are not limited to, ultraviolet radiation absorbers, antioxidants, organic or inorganic colorants (e.g., dyes or pigments), stabilizers, fragrances, plasticizers, anti-microbial agents, flame retardants, antifouling compounds, and combinations thereof. The amount of each optional additive is generally no more than about 15 wt-% of the material used to form a given elastic tag component, often no more than 5 wt-% of the material used to form a given elastic tag component.

The elastic tags of the present invention may have overall dimensions that vary depending on the intended use. Typically, the overall thickness of an elastic tag, the thickness of first tag portion 11, the thickness of second tag portion 12 and the thickness of third tag portion 18 (when present) may each vary independently from one another based on a number of factors including, but not limited to, the materials used, and a particular application or use. Typically, each of (i) the overall thickness of an elastic tag, (ii) the thickness of first tag portion 11, (iii) the thickness of second tag portion 12 and (iv) the thickness of third tag portion 18 (when present) is less than about 0.64 mm (0.25 in more typically, less than about 0.32 mm (0.12 in.), and more typically, between about 0.01 mm (0.4 mils) to about 0.32 mm (0.12 in.).

As shown in FIGS. 1A-1D and 4A-4C, elastic tags of the present invention may have a variety of overall shapes. It should be understood that the elastic tags of the present invention may have any desired shape. Further, elastic loops (e.g., first closed loop 13 and second closed loop 16) of the elastic tags may have any dimensions, sizes and shapes. In some embodiments, the elastic loop component (e.g., first closed loop 13 and second closed loop 16) has an overall width that is equal to or less than an overall width of first tag portion 11. In other embodiments, one or more of the elastic loop components (e.g., first closed loop 13 and second closed loop 16) may have an overall width that is greater than an overall width of first tag portion 11.

Although the elastic tags of the present invention may have any desired dimensions, typically, elastic tags of the present invention have an overall length of less than about 61 centimeters (cm) (24 in.), more typically, less than about 30.5 cm (12 in.), and more typically, between about 7.6 mm (3.0 in.) to about 30.5 cm (12 in.). Typically, elastic tags of the present invention have an overall width of less than about 15.2 cm (6.0 in.), more typically, less than about 7.6 cm (3.0 in.), and more typically, between about 12.7 mm (0.5 in.) to about 7.6 cm (3.0 in.).

The present invention is further directed to methods of making the disclosed elastic tags. In one exemplary method of making an elastic tag, the method comprises integrally attaching a second tag portion to a first tag portion so that the second tag portion extends beyond a first edge portion of an outer edge of the first tag portion, the second tag portion comprising a first closed loop having a loop inner periphery, at least a portion of the loop inner periphery being positioned a distance from the first tag portion and comprising an elastomeric material; and at least one step selected from the steps of: (1) forming at least one slotted opening within the first tag portion so as to be in communication with an outer periphery of the first tag portion, and (2) providing a second closed loop of elastic material along the outer periphery of the first tag portion.

First tag portion 11 may be formed from any of the above-described materials using any conventional method. Suitable methods include, but are not limited to, a sheet or film forming step (e.g., a papermaking step or a film extrusion step); a molding step such as an injection molding step; a coating step (e.g., to provide a writable or printable surface on a base substrate); a lamination step (e.g., to bond two or more layers to one another); a cutting step (e.g., stamping) so as to form outer periphery 111, any slotted openings 15, and second closed loop 16 (when present); or any combination of the above steps.

Second tag portion 12 and third tag portion 18 (when present as a separate component from first tag portion 11) may be formed using any of the above-described method steps for forming first tag portion 11. Second tag portion 12 and third tag portion 18 (when present as a separate component from first tag portion 11) may be integrally attached to first tag portion 11 by any known method. Suitable attachment methods include, but are not limited to, an adhesive bonding step using a separate adhesive component; an adhesive bonding step wherein one or materials used to form first tag portion 11, second tag portion 12 and/or third tag portion 18 (when present as a separate component from first tag portion 11) act as an adhesive component; a molding step such as an injection molding step (e.g., first tag portion is positioned within a mold and second tag portion material is introduced into the mold); a lamination step (e.g., to bond one or more layers of first tag portion 11 to one or more layers of second tag portion 12); or any combination of the above steps.

In one desired embodiment, second tag portion 12 (and/or third tag portion 18) is integrally attached to first tag portion 11 via an adhesive, wherein the adhesive is (i) a component other than the materials used to form first tag portion 11, second tag portion 12 or third tag portion 18, (ii) a component used to form first tag portion 11, second tag portion 12 or third tag portion 18, or (iii) both (i) and (ii).

The methods of forming an elastic tag may further comprise one or more of the following method steps:

(1) forming first closed loop 13 within second tag portion 12 prior to or after an integrally attaching step;

(2) forming at least one slotted opening 15 within first tag portion 11;

(3) cutting one or more slotted opening 15 within first tag portion 11;

(4) forming at least one slotted opening 15 within first tag portion 11 simultaneously with a step of forming first tag portion 11;

(5) providing a second closed loop of elastic material along outer periphery 111 of first tag portion 11;

(6) integrally attaching third tag portion 18 to first tag portion 11, wherein third tag portion 18 comprises second closed loop 16, wherein second closed loop 16 comprises an elastic material and has a second closed loop inner periphery 161, and at least a portion of second closed loop inner periphery 161 is positioned a distance from first tag portion 11;

(7) applying indicia onto first tag portion 11; and

(8) packaging one or more elastic tags.

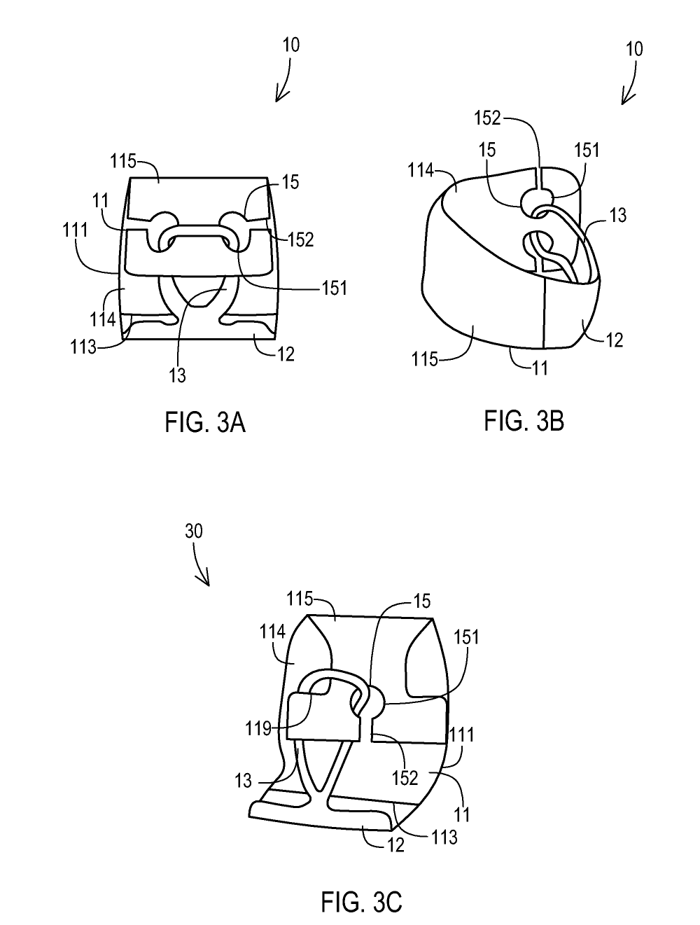

The present invention is even further directed to methods of using the disclosed elastic tags. In one exemplary method of using an elastic tag, the method comprises surrounding at least a portion of an article with an elastic tag comprising a first tag portion having a first closed loop integrally attached thereto; and connecting the first closed loop of the elastic tag with (i) one or more slotted opening within the first tag portion or (ii) a second closed loop of elastic material along an outer periphery of the first tag portion. FIGS. 3A-3C depict frontal and side views of the exemplary elastic tags of FIGS. 1A and 1C when a closed loop portion of a given exemplary elastic tag is connected to at least one slotted opening of the given exemplary elastic tag.

As shown in FIGS. 3A and 3B, exemplary elastic tag 10 may be used to surround a portion of an article (not shown) so that the article is positioned along upper surface 114 of exemplary elastic tag 10. In this embodiment, first closed loop 13 of elastic tag 10 is inserted into and through channels 152 on opposite sides of first tag portion 11 so as to rest within loop containment reservoirs 151 located within an interior portion (e.g., away from outer periphery 111) of first tag portion 11. It should be noted that although not shown, exemplary elastic tag 10 could be used to surround a portion of an article (not shown) so that the article is positioned along lower surface 115 of exemplary elastic tag 10. In other words, upper surface 114 could be facing away from the enclosed/surrounded article.

FIG. 3C provides another view of the use of an elastic tag of the present invention. In this exemplary embodiment, exemplary elastic tag 30 may be used to surround a portion of an article (not shown) so that the article is positioned along upper surface 114 of exemplary elastic tag 30. In this embodiment, first closed loop 13 of elastic tag 30 is inserted into and through a single channel 152 on a side of first tag portion 11 positioned away from first closed loop 13 so as to rest within loop containment reservoir 151 located within an interior portion (e.g., away from outer periphery 111) of first tag portion 11. In this embodiment, a single slotted opening 15 is used in combination with a ledge portion 119 of first tag portion 11 to temporarily secure first closed loop 13 to first tag portion 11. As discussed above, it should be noted that although not shown, exemplary elastic tag 30 could be used to surround a portion of an article (not shown) so that the article is positioned along lower surface 115 of exemplary elastic tag 30 (i.e., upper surface 114 could be facing away from the enclosed/surrounded article).

In other embodiments, a separate connecting member may be used to surround a given article. One exemplary embodiment utilizing a separate connecting member is shown in FIG. 5. As shown in FIG. 5, exemplary elastic tag 60 of FIG. 4B is configured so as to surround an article (not shown), wherein a connecting member 61 is used to join first closed loop 13 to second closed loop 16. Connecting member 61 may comprise, for example, a plastic molded component, a metal component, a ceramic component, an elastomeric component, or any other type of component as long as connecting member 61 has enough structural integrity to connect first closed loop 13 to second closed loop 16.

The elastic tags of the present invention may be used to surround a variety of articles and attach the article to another object or to itself. In one desired embodiment, the elastic tags of the present invention are used to control the position of a drape along a piece of medical equipment. Suitable pieces of medical equipment include, but are not limited to, a C-arm apparatus, a microscope, a surgical knife system, endoscopy equipment, an operating room table, a patient, a light, or a lamp.

The method of using the elastic tags of the present invention may further comprise one or more of the following steps:

(1) connecting first closed loop 13 with one or more slotted opening 15 within first tag portion 11; and

(2) connecting first closed loop 13 with second closed loop 16 of elastic material positioned along outer periphery 111 of first tag portion 11.

The present invention is described above in a manner, which is not to be construed in any way as imposing limitations upon the scope of the invention. On the contrary, it is to be clearly understood that resort may be had to various other embodiments, modifications, and equivalents thereof which, after reading the description herein, may suggest themselves to those skilled in the art without departing from the spirit of the present invention and/or the scope of the appended claims.

While the specification has been described in detail with respect to specific embodiments thereof, it will be appreciated that those skilled in the art, upon attaining an understanding of the foregoing, may readily conceive of alterations to, variations of, and equivalents to these embodiments. Accordingly, the scope of the present invention should be assessed as that of the appended claims and any equivalents thereto.

What is claimed is:

1. An elastic tag comprising:
a first tag portion having an outer periphery extending along an outer edge of the first tag portion; and
a second tag portion integrally attached to said first tag portion and extending beyond a first edge portion of the outer edge of the first tag portion, said second tag portion comprising a first closed loop having a loop inner periphery, at least a portion of said loop inner periphery being positioned a distance from said first tag portion and comprising an elastomeric material,
wherein said first tag portion further comprises (a) at least one slotted opening positioned along and in communication with said outer periphery, (b) a second closed loop of elastic material, or (c) both (a) and (b).

2. The elastic tag of claim 1, wherein a substantial portion of said second tag portion comprises an elastomeric material.

3. The elastic tag of claim 1, wherein at least 90 wt-% of said second tag portion comprises an elastomeric material.

4. The elastic tag of claim 1, wherein said first tag portion comprises at least two slotted openings positioned along and in communication with said outer periphery.

5. The elastic tag of claim 4, wherein said at least two slotted openings comprise one or more pairs of slotted openings, wherein each slotted opening within a given pair of slotted openings is positioned a substantially equal distance from said first closed loop.

6. The elastic tag of claim 5, wherein said at least two slotted openings comprise two pairs of slotted openings, wherein (i) each slotted opening within a first pair of slotted openings is positioned a first distance from said first closed loop, (ii) each slotted opening within a second pair of slotted openings is positioned a second distance from said first closed loop, and (iii) said first distance is greater than said second distance.

7. The elastic tag of claim 4, wherein said outer periphery of said first tag portion comprises (i) a second edge portion opposite said first edge portion, (ii) opposite side edges connecting said second edge portion to said first edge portion, and (iii) at least one slotted opening positioned along each of said opposite side edges.

8. The elastic tag of claim 4, wherein said outer periphery of said first tag portion comprises (i) a second edge portion opposite said first edge portion, (ii) opposite side edges connecting said second edge portion to said first edge portion, (iii) at least one slotted opening positioned along said second edge portion, and (iv) at least one slotted opening positioned along one or both of said opposite side edges.

9. The elastic tag of claim 1, wherein said first tag portion comprises a second closed loop of elastic material.

10. The elastic tag of claim 9, wherein said elastic tag comprises a third tag portion integrally attached to said first tag portion and extending beyond a third edge portion of the outer edge of the first tag portion, said third tag portion comprising the second closed loop, said second closed loop having a second closed loop inner periphery, at least a portion of said second closed loop inner periphery being positioned a distance from said first tag portion and comprising an elastomeric material.

11. The elastic tag of claim 10, wherein said second and third tag portions are positioned along opposite edges of said outer periphery.

12. The elastic tag of claim 11, wherein said first tag portion has a substantially rectangular shape having a first tag portion length that is greater than a first tag portion width, and said first and second closed loops are positioned along opposite edges of the first tag portion separated from one another by a substantial portion of the first tag portion length.

13. The elastic tag of claim 12, wherein said outer periphery of said first tag portion comprises (i) opposite side edges extending between said first and second closed loops, and (ii) at least one slotted opening positioned along each of said opposite side edges.

14. The elastic tag of claim 9, further comprising a connecting member operatively adapted to connect said first closed loop to said second closed loop.

15. The elastic tag of claim 14, wherein said connecting member is attached to said first closed loop and is operatively adapted to connect to said second closed loop.

16. The elastic tag of claim 1, wherein said first tag portion comprises a sheet of inelastic material.

17. The elastic tag of claim 16, wherein said inelastic material comprises paper, a polymeric film material, a polymeric foam material, an electrically conductive material, a ceramic material, a glass material, and any combinations thereof.

18. The elastic tag of claim 1, wherein said first tag portion comprises a sheet of material having opposite outer major surfaces, at least one of said opposite outer major surfaces being capable of accepting and displaying indicia thereon.

19. A drape in combination with the elastic tag of claim 1.

20. A method of using an elastic tag, said method comprising:
surrounding at least a portion of an article with the elastic tag; and
connecting the first closed loop with (i) one or more slotted opening within the first tag portion or (ii) a second closed loop of elastic material along the outer periphery of the first tag portion.

21. The method of claim 20, further comprising:
attaching the elastic tag and the portion of the article to at least a portion of a piece of medical equipment.

22. The method of claim 21, wherein the article comprises a drape.
